(19) United States
(12) Patent Application Publication
(10) Pub. No.: US 2025/0143866 A1
(43) Pub. Date: May 8, 2025

(54) BIFURCATED STENT GRAFT WITH PATIENT-SPECIFIC FENESTRATIONS

(71) Applicant: W. L. Gore & Associates, Inc., Newark, DE (US)

(72) Inventors: Kristin Brune, Scottsdale, AZ (US); Paul Morgan, Gilbert, AZ (US); Martin J. Sector, Phoenix, AZ (US)

(21) Appl. No.: 18/937,748

(22) Filed: Nov. 5, 2024

Related U.S. Application Data
(60) Provisional application No. 63/596,983, filed on Nov. 8, 2023.

(51) Int. Cl. A61F 2/07; A61F 2/06

(52) U.S. Cl. CPC A61F 2/07; A61F 2002/065; A61F 2240/002; A61F 2250/0098

(57) ABSTRACT

Devices, systems and methods for patient-specific, custom fenestrated endoprostheses. Various examples relate to abdominal aorta stent-grafts with custom fenestration features for perfusing the renal arteries, mesenteric, and/or celiac trunk artery.

### CROSS-REFERENCE TO RELATED APPLICATION

This application claims the benefit of Provisional Application No. 63/596,983, filed Nov. 8, 2023, which is incorporated herein by reference in its entirety for all purposes.

### BACKGROUND

Custom endoprostheses (e.g., stent grafts for aneurysmal repair) are generally patient-specific devices tailored to a particular individual's anatomy. In this context, “custom” solutions are not accomplished by unique combinations of existing devices, and are generally manufactured in view of patient anatomy to treat vascular areas that often include one or more side branches (e.g., with regard to the aorta, treatment of thoracoabdominal, juxtarenal, and suprarenal aneurysms, as well as aortic arch repair). While in a broad sense, customization may simply involve changing endoprosthesis dimensions (e.g., diameter along the length of the endoprosthesis), as discussed in the context of this patent specification, customization relates to the provision of fenestrations in endoprostheses that are customized in a manner that accommodates a specific patient's or group of patients' anatomical configuration(s).

### SUMMARY

Customized fenestrations in endoprostheses provide patient-specific device options to health care providers. In some embodiments, the fenestrations are reinforced with additional material, such as additional material on the surface of the endoprosthesis, or additional material forming an external and/or internal tube (e.g., film tube). In some embodiments, one or more fenestrations are marked or otherwise associated with radiopaque material, such as a ring, pattern of markers, or other radiopaque indicators. In some embodiments, removable guidewire tubes, cannulation fibers, cannulation wires, or other features are placed in one or more of the fenestrations to facilitate pre-cannulation of the fenestrations in association with device delivery (e.g., pre-cannulation with one or more guidewires). In some embodiments, the endoprosthesis includes a substantial length that is unsupported to provide sufficient area for the placement (e.g., formation at a remote location, such as a manufacturing location, or on-site at the location of implantation) of custom, patient-specific fenestration features. In some embodiments, the endoprosthesis delivery system components associated with the endoprosthesis (e.g., constraining sleeve components) may be altered to better suit orientation and positioning of the fenestration(s) (e.g., intermediate deployment technologies, such as multiple releasable seams for deployment of one or more portions of the endoprosthesis to one or more intermediate diameters that are less than a final, fully deployed diameter.

According to one example (“Example 1”), an endoprosthesis article of manufacture includes a main body including a crown portion, a hip portion, and an intermediate portion between the crown portion and the hip portion, the crown and hip portions being supported by one or more frame elements and the intermediate portion being unsupported by any frame element; a first leg extending from the hip portion of the main body; and a second leg extending from the hip portion of the main body adjacent to the first leg.

According to another example (“Example 2”) further to Example 1, the intermediate portion is configured for subsequent formation of one or more patient-specific fenestration features.

According to another example (“Example 3”) further to Example 1, wherein the one or more frame elements have an undulating shape defining a maximum amplitude and the intermediate portion has a height that is at least twice the maximum amplitude, or, optionally, at least five times the maximum amplitude, or optionally at least ten times the maximum amplitude.

According to another example (“Example 4”) further to Example 3, the one or more patient-specific fenestration features are at pre-selected locations corresponding to one or more abdominal aortic branch vessels of a specific patient.

According to another example (“Example 5”) further to any preceding Example, the crown portion is configured to anchor the endoprosthesis in the abdominal aorta at a location superior to the renal arteries.

According to another example (“Example 6”), an endoprosthesis includes a main body including a crown portion, a hip portion, and an intermediate portion between the crown portion and the hip portion, the crown and hip portions being supported by one or more frame elements, the intermediate portion having one or more fenestration features associated with the intermediate portion, the one or more fenestration features including an aperture through a graft element of intermediate portion and a port lining extending through the aperture; a first leg extending from the hip portion of the main body; and a second leg extending from the hip portion of the main body adjacent to the first leg.

According to another example (“Example 7”) further to Example 6, the intermediate portion is unsupported by any frame element.

According to another example (“Example 8”) further to Example 6, wherein the intermediate portion is supported by a frame element formed so as not to obstruct the fenestration feature.

According to another example (“Example 9”) further to Examples 6 or 8, the intermediate portion is supported by one or more frame elements having an undulating shape that has a varying amplitude and/or frequency to accommodate the fenestration feature, the undulating shape of the one or more frame elements of the intermediate portion being different from the one or more frame elements supporting the crown and the hip portions.

According to another example (“Example 10”) further to any one of Examples 6 to 9, the port lining is a tubular member formed of a film material and that is free of any frame element.

According to another example (“Example 11”) further to any one of Examples 6 to 9, the aperture and/or the port lining are reinforced with one or more of the following: polymeric ring elements, fiber elements, wire elements, and combinations thereof.

According to another example (“Example 12”) further to any one of Examples 6 to 11, the fenestration feature includes a radiopaque marker selected from one or more of the following: a circumferential marker, a dot marker, an impregnated marker, and combinations thereof.

According to another example (“Example 13”) a method of making an endoprosthesis includes obtaining patient-specific branch vessel location data; and forming one or more fenestration features in an unsupported, intermediate portion of an endoprosthesis article of manufacture based upon the patient-specific branch vessel location data.

According to another example (“Example 14”) further to Example 13, the method further includes providing the intermediate portion with a frame element supporting the intermediate portion, the frame element being configured so as not to obstruct the one or more fenestration features.

According to another example (“Example 15”) further to Examples claim 13 or 14, the endoprosthesis article of manufacture is made prior to the patient-specific branch vessel location data being obtained.

According to another example (“Example 16”) further to any one of Examples 13 to 15, wherein forming the one or more fenestration features includes forming an aperture through a graft element of intermediate portion and disposing a port lining through the aperture and coupling the port lining to the graft element of the intermediate portion.

According to another example (“Example 17”) further to any one of Examples 13 to 16, wherein forming the one or more fenestration features includes associating a radiopaque marker selected from one or more of the following with the fenestration feature: a circumferential marker, a dot marker, an impregnated marker, and combinations thereof.

According to another example (“Example 18”) further to any one of Examples 13 to 17, wherein forming the one or more fenestration features includes forming fenestration features corresponding to at least three aortic branch vessels of a specific patient.

According to another example (“Example 10”) further to Examples 18, wherein the aortic branch vessels include a first renal artery, a second renal artery, a mesenteric artery, and/or a celiac trunk artery.

According to another example (“Example 10”) further to any one of Examples 13 to 19, wherein forming the one or more fenestration features includes reinforcing the aperture with one or more of the following: polymeric ring elements, fiber elements, wire elements, and combinations thereof.

The various examples addressed in this patent specification should not be read to limit or otherwise narrow the scope of any of the inventive concepts that are otherwise provided. While multiple examples are disclosed, still other embodiments will become apparent to those skilled in the art from the description and drawings, which show and describe illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows an endoprosthesis, according to some embodiments.

FIG. 2 shows an enlarged view of a portion of the endoprosthesis of FIG. 1, according to some embodiments.

FIGS. 3 to 5 show examples of radiopaque markers of the endoprosthesis of FIG. 1.

FIG. 6 shows an endoprosthesis article, according to some embodiments.

FIG. 7 illustrates a method of manufacture for the endoprosthesis of FIG. 1, according to some embodiments.

FIG. 8 illustrates another method of manufacture for the endoprosthesis of FIG. 1, according to some embodiments.

FIG. 9 shows an endoprosthesis configuration, according to some embodiments.

FIG. 10 shows another endoprosthesis configuration, according to some embodiments.

FIG. 11 shows another endoprosthesis configuration according to some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “fenestration” or “fenestration feature” as used herein is meant to designate an opening or openings of sufficient size in an endoprosthesis to permit substantial perfusion, or flow, into one or more branch vessels (e.g., including any of those described herein) from the endoprosthesis.

### Overview

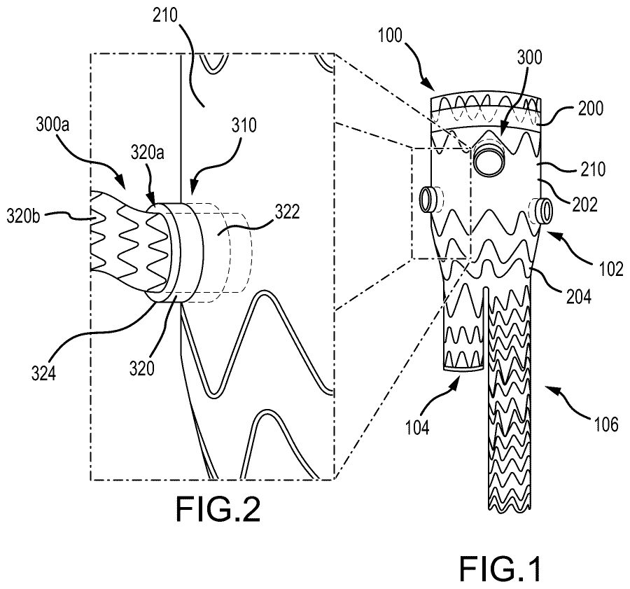

The various designs addressed in this patent specification relate to an endoprosthesis 100 including one or more graft components and one or more support components coupled to the graft component(s). As shown in FIG. 1, the endoprosthesis 100 includes a main body 102. The main body 102 is terminally bifurcated with a larger proximal inlet and two distal outlets defined by two legs, such as a short leg and a long leg. Though not shown, the main body 102 may alternatively be formed with a larger, single proximal inlet and a larger, single distal outlet, without defining separate legs or branches at the distal end, for example. As shown, the main body 102 bifurcates into a first leg 104, and a second leg 106. The first leg 104 is positioned adjacent the second leg 106. As shown, the legs extend in substantially the same general direction, although diverging legs or other arrangements are contemplated. While the first and second legs 104, 106 may be of differing lengths, they may also have the same length. In some embodiments, one or both of the legs 104, 106 may be a stub, or even simply an opening without any substantial length extending from the main body 102.

As shown, the endoprosthesis 100 is configured to be endovascularly implanted in (e.g., using a catheter delivery system, not shown), and to treat the abdominal aorta. However, configurations for treating other vasculature (e.g., the aortic arch) are also contemplated. In various examples, the endoprosthesis 100 is used to treat an aortic aneurysm. In general terms, in some embodiments, the endoprosthesis 100 is configured to help direct blood through the aorta while protecting the walls of the aorta from further pressure from the blood flow. In other words, the endoprosthesis 100 acts to carry the blood flow to relieve pressure on damaged, weakened or diseased portions of the vasculature, such as the aorta. For example, the legs 104, 106 may be configured to be directed into (either directly, or through an extension graft) into the iliac arteries with the main body located in the abdominal aorta. The endoprosthesis 100 includes a framework component comprised of one or more frame elements (e.g., circumferential and/or helical stent windings, braidings, knits or other stent forms) and a graft component comprised of one or more graft elements (e.g., extruded, wrapped, braided, knit or otherwise configured). Examples of suitable materials for the framework and graft components are described in subsequent sections.

As shown, the main body 102 includes a crown portion 200, an intermediate portion 202, and a hip portion 204. The crown portion 200 generally includes one or more crown frame elements (e.g., circumferential and/or helical stent supports) configured for anchoring the crown portion 200, and thus the endoprosthesis 100 more generally, in the vasculature (e.g., abdominal aorta). The one or more crown frame elements may be evenly spaced from one another (e.g., evenly-spaced, separate circumferential rings, or evenly-spaced, helically wound turns), according to some examples. The crown portion 200 also includes a crown graft element. The intermediate portion 202 also includes an intermediate graft element 210, but as shown is free of frame elements, also described as unstented or unsupported by any secondary frame elements. Similar to the crown frame elements, the one or more hip frame elements may be evenly spaced from one another (e.g., evenly-spaced separate circumferential rings, or evenly-spaced, helically wound turns), according to some examples. Though shown with even spacing, any combination of even and uneven spacing between the various frame elements in any of the different portions described above or below is contemplated.

The intermediate portion 202 has a substantial height in the axial direction, that is substantially greater than the frame element spacing of the crown and hip portions. In some examples, the frame element(s) of the crown and hip portions 202, 204 have an undulating shape defining a maximum amplitude and the intermediate portion 202 has a height that is at least twice the maximum amplitude, or, optionally, at least five times the maximum amplitude, or optionally at least ten times the maximum amplitude, although a variety of dimensions are contemplated. For example, the intermediate portion 202 may have a height that is 1 cm, 2 cm, 10 cm, 15 cm, 20 cm or any value or range between any of the foregoing, although any of a variety of dimensions are contemplated.

The intermediate graft element 210 may be provided with additional material or thickness to make it more resilient to collapse, buckling, invagination, etc. The hip portion 204 also includes one or more hip frame elements (e.g., circumferential and/or helical stent supports) configured for supporting a hip graft element. Similarly, the first leg 104 includes a frame element and graft element, and the second leg includes a frame element and graft element. One or more of the various frame elements described above may be continuously formed with one another, integral, interconnected, or may be formed separate, unconnected components as desired. And, one or more of the various graft elements described above may be continuously formed with one another, integral, interconnected, or as separate components as desired.

In various examples, the main body 102 is configured to be customized to a patient anatomy, and anchored therein (e.g., within the abdominal aorta), the main body including one or more fenestration features 300 formed at custom locations corresponding to one or more branch vessels (e.g., aortic) of a patient, or group of patients. These fenestration features are pre-selected according to patient anatomy, such as at locations corresponding to one or more abdominal aortic branch vessels of a specific patient. For example, the main body 102 may be anchored at a location that is superior to a location where the renal arteries (RA) branch from the aorta, and includes fenestration features 300 for permitting perfusion of those renal arteries. In some examples, the main body 102 is anchored at a location superior, or proximate to the superior mesenteric artery and/or celiac trunk artery and includes fenestration features 300 for permitting perfusion for one or both of those aortic branch vessels. For example, the fenestration features 300 may be configured a first renal artery, a second renal artery, a mesenteric artery, and/or a celiac trunk artery and so forth.

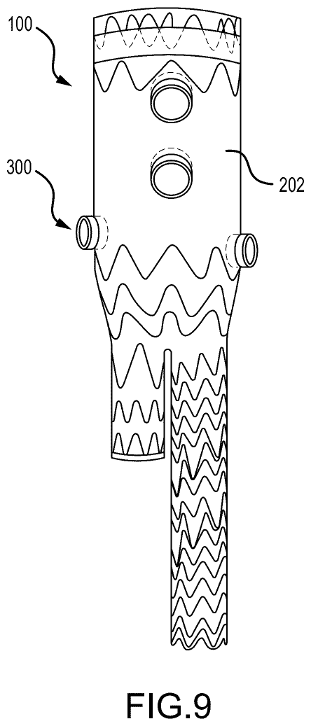

As shown, the one or more fenestration features 300 may be located within the intermediate portion 202. As described in greater detail below, the intermediate portion 202 may be left free of any frame elements, or may have customized frame elements (see FIG. 11) added thereto that correspond to the positioning of the fenestration features 300 (e.g., the frame element positions/configurations are not determined until after positioning of the fenestration features 300 is determined). The intermediate portion 202 may be supported by one or more frame elements formed so as not to obstruct the fenestration features 300. For example, as shown in FIG. 11, frame elements having custom shapes, such as varying amplitudes and/or frequencies in their undulating shapes (peaks and valleys), may be employed to accommodate the fenestration features 300. The frame elements may be relatively close to the fenestration features 300, thereby also providing support to the fenestration features 300. And, as shown in FIG. 9, the intermediate portion 202 may be lengthened or adjusted in overall area (e.g., by removing the presence of one or more stent rows or lengthening the overall length of the intermediate portion 202). By adjusting the overall area of the intermediate portion 202, the intermediate portion 202 may be configured to accept or be configured with greater or fewer fenestration features 300 and/or locations, including locations corresponding to those vascular branches referenced herein.

### Fenestration Features

FIG. 2 is a closeup, or enlarged view of one of the fenestration features 300, where FIG. 2 shows a first fenestration feature 300a of the one or more fenestration features 300. The fenestration features 300 are generally patient-specific, in that they have been located and configured to support a specific patient, or a specific group of patients characterized as being within a range of common anatomy characteristics (e.g., having similarly located and sized vessels and branch vessels at a desired treatment site). The various fenestration features 300 may be similarly configured, and thus may be described cumulatively with regard to the first fenestration feature 300a (although differently configured fenestration features—differently sized, shaped, supported—e.g., nitinol frame, unsupported, etc. are also contemplated). As shown in FIG. 2 the first fenestration feature 300a includes an aperture 310 formed into the intermediate graft element 210 at a patient-specific location corresponding to a branch vessel (e.g., aortic branch vessel, such as a renal artery). The first fenestration feature 300a includes a port lining 320 received in, and coupled within a boundary of the aperture 310. Generally, the port lining 320 may be formed as cylindrical tube of material, or is otherwise formed as a tubular member. The port lining 320 may be formed of any of a variety of materials, including any of the graft materials described herein. As shown, the port lining 320 extends through the aperture 310, defining an inner portion 322 on an interior side of the aperture 310 and an outer portion 324 on an outer side of the aperture 310. Although the port lining 320 may have an inner portion 322 and an outer portion 324, in some embodiments the port lining 320 only extends from the outer surface (only has an outer portion 324) or the port lining 320 only extends from the inner surface (only has an inner portion 322). The port lining 320 may be flexible, and formed as a right cylinder (e.g., being free to flex or bend, but not be formed with a bend or curve). If desired, the port lining 320 may have one or more portions that are more resilient, or resistant to flexing, than other portions and/or the port lining 320 may have pre-formed bend(s), taper(s), or other shape. In some embodiments, the port lining 320 has a lumen 320a. The lumen 320a may be adapted to accept a guidewire. As shown in FIG. 2, the port lining 320 can be adapted to accept placement of a side branch component 320b (e.g., a stent graft endoprosthesis for perfusing one or more branch vessels).

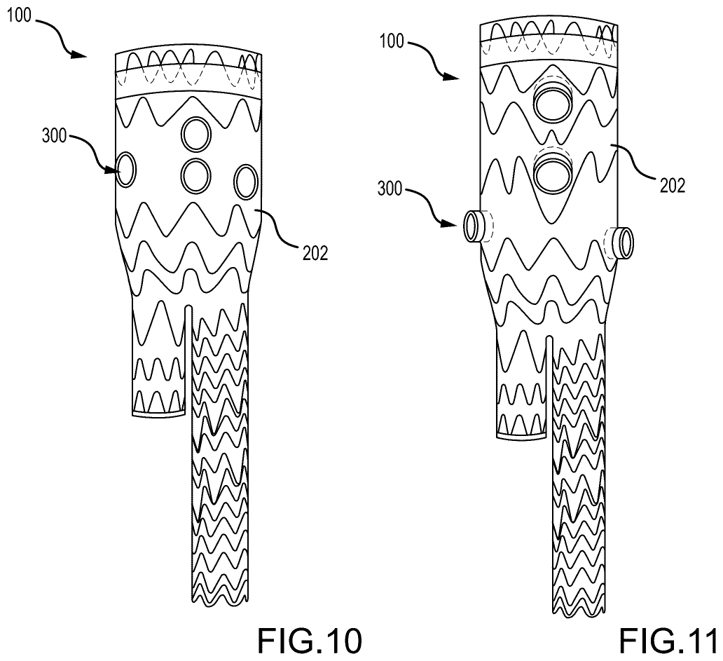

In some embodiments, the port lining has an overall length of between 1 and 10 mm, such as approximately 5 mm, although a variety of lengths are contemplated. Although port lining is shown in association with each of the apertures of the various fenestration features 300, it should be understood that is not necessarily the case in all embodiments, and that fenestration features 300 without the port lining component are also contemplated, for example as shown in FIG. 10. Additionally or alternatively, various reinforcements may be provided to reinforce the fenestration features (e.g., the aperture 310 and/or port lining 320), including one or more of the following: polymeric ring elements, fiber elements, wire elements, and combinations thereof. In different terms, the port lining 320 may be free of any structural reinforcement members (e.g., stents, rings, or other reinforcement) or may be structurally reinforced by one or more structural reinforcement members as indicated generally in FIG. 4 by the features in broken lines. In some embodiments, the port lining 320 is reinforced by a collapsible reinforcement element.

Generally, the port lining 320 may assist with any of a variety of functions. For example, the port lining 320 may extend partially into the branch vessel to which the aperture 310 is aligned. The port lining 320 may act as additional seal material to assist with sealing to the branch vessel. Additionally or alternatively, the port lining 320 may serve as an anchor point and/or sealing feature for use with a branch endoprosthesis. For reference, any of a variety of branch endoprostheses are contemplated, including branch stent-grafts. Examples of suitable branch stent graft include, for example, a suitably configured GORE® VIABAHN® VBX Balloon Expandable Endoprosthesis.

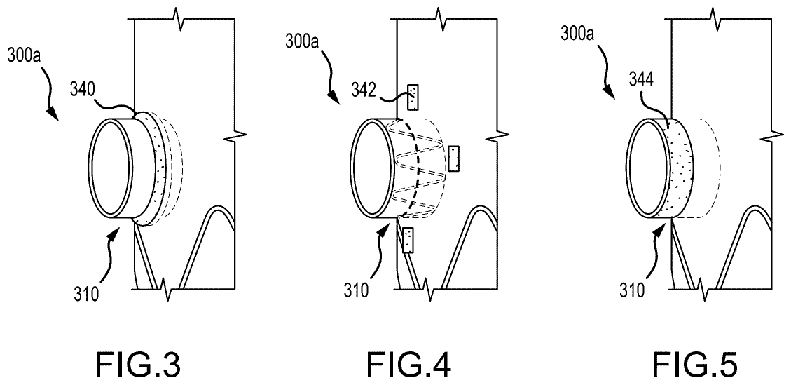

The one or more fenestration features 300 also optionally include radiopaque markings. For example, FIG. 3 shows a circumferential radiopaque marker 340 that encircles, borders, surrounds, or is otherwise proximate to the aperture 310. FIG. 4 shows a radiopaque marker 342 in the form of a plurality of dots or discrete markings (e.g., four) about the aperture 310. FIG. 5 shows a radiopaque marker 344 in the form of radiopaque material impregnated, or otherwise associated with the port lining 320. Although some examples are shown, any of a variety of radiopaque marker configurations are contemplated. For example, the fenestration features 300 may include a radiopaque marker selected from one or more of the following: a circumferential marker, a dot marker, an impregnated marker, and combinations thereof.

In various embodiments, the endoprosthesis is formed to include up to one, two, three, four, or five or more fenestration features 300 in the intermediate portion 202, such as fenestration features corresponding to custom, patient-specific locations for one or both of the renal arteries (RA), the superior mesenteric artery, the celiac trunk, and/or accessory renal(s), for example. In some embodiments, the endoprosthesis may be fenestrated, or include fenestration features, suitable for perfusing multiple renals (e.g., 3), a superior mesenteric artery, and an associated method of treatment may include placing the endoprosthesis below the celiac vessel, for example.

### Methods of Forming Custom Fenestration Features

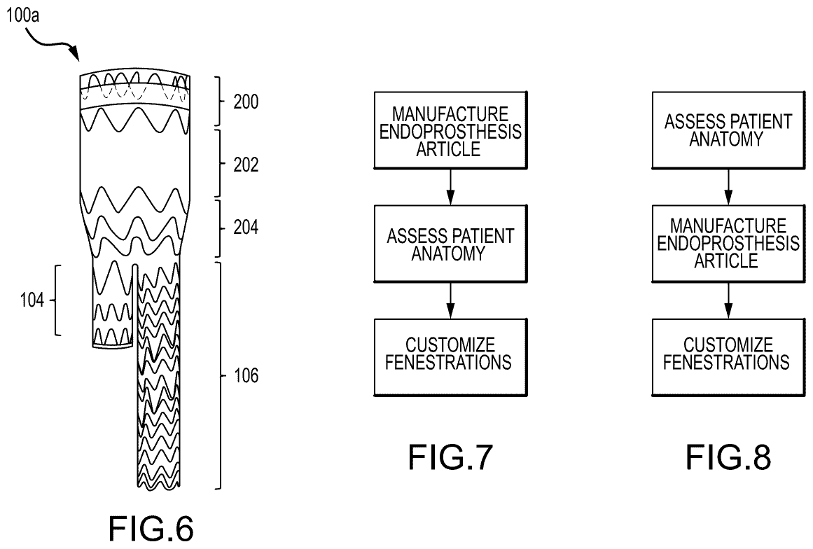

FIG. 6 shows an endoprosthesis article 100a that is a precursor prior to formation of the fenestration features 300 in the endoprosthesis 100 of FIG. 1, or a finished medical device for implantation. The endoprosthesis article 100a includes the crown portion 200, intermediate portion 202, hip portion 204, and first and second legs 104, 106 as previously described in association with FIG. 1. As shown, the intermediate portion 202 of the endoprosthesis article 100a is free of one or more fenestration features to be subsequently formed. In other words, the intermediate portion 202 may be completely free of fenestration features, or may have some number of fenestration features excluding one or more fenestration features that are to be subsequently formed in the intermediate portion. In particular, the endoprosthesis article 100a represents the manufacturing state of the Endoprosthesis 100 prior to formation of the fenestration features 300. For reference, the fenestration features 300 may be formed at a remote location (e.g., a manufacturing site or preparation site) or at a surgical location, either before or after implantation, or at another location as desired. The fenestration features 300 may be formed by a manufacturer, a health care provider, or other entity as appropriate. As described below, the fenestration features 300 may be formed immediately after gathering patient anatomy data, or at some time (e.g., hours, days, months, years) after gathering patient anatomy data.

FIG. 7 shows a first method of forming, also described as a method of making, the customized endoprosthesis 100. In various examples, the method includes forming one or more fenestration features in an unsupported, intermediate portion of an endoprosthesis article of manufacture based upon patient-specific branch vessel location data that has been obtained. Any number of fenestration features may be formed depending upon the particular device application. For example, the method may include forming fenestration features corresponding to at least one branch vessel (e.g., aortic branch vessel) of a specific patient, at least two of such branch vessels, at least four of such branch vessels, at least five of such branch vessels and so forth.

As indicated in FIG. 7, the method includes manufacturing the endoprosthesis article 100a. This may be done months, weeks, days, hours, or immediately prior to the subsequent step, which is assessing patient anatomy. This may require obtaining patient-specific branch vessel location data. For example, during this step, patient anatomy data may be gathered (e.g., using CT scanning, ultrasound, or other appropriate assessment technology). Once the anatomy data is gathered, either for a single patient, or a grouping of patients, the one or more fenestration features 300 are custom-formed in the intermediate portion 202 of the endoprosthesis article 100a. For example, using the anatomy data, fenestration features 300 may be formed in the intermediate portion 202 at locations corresponding to one or more branch vessels, including any of those previously described. As part of forming one or more fenestration features 300, the method may also include coupling port lining 300 to a graft element of the intermediate portion 202. For example, after forming an aperture (e.g., aperture 310) through the graft element of intermediate portion 202, port lining 300 may be disposed through the aperture and then the port lining may be coupled to the graft element of the intermediate portion 202. If desired, a radiopaque marker (e.g., radiopaque maker 342) may be associated with the fenestration feature (aperture and/or port lining), including those selected from one or more of the following: a circumferential marker, a dot marker, an impregnated marker, and combinations thereof.

If desired, in an optional step, a custom framework (FIG. 11) may be added in a manner that does not interfere with the functionality fenestration features 300. The foregoing process may be advantageous from the standpoint that, by pre-manufacturing the endoprosthesis article 100a (e.g., days, weeks, months, etc.) ahead of gather the patient-specific data and/or forming the fenestration features, lead time can be significantly reduced in the process of providing custom-made endoprostheses.

FIG. 8 shows an alternative method of forming the customized endoprosthesis 100. As shown in FIG. 8, the afore-mentioned anatomy data is gathered prior to forming the endoprosthesis article 100a. The endoprosthesis article 100a is then manufactured, with the fenestration features 300 being prepared in the intermediate portion 202 of the endoprosthesis article 100a. The fenestration features 300 may be formed as part (e.g., as part of single manufacturing process) of manufacturing the endoprosthesis article 100a. Alternatively, the fenestration features 300 may be incorporated into the endoprosthesis article 100a months, weeks, days, hours, following manufacture of the endoprosthesis article 100a.

### Materials

The materials used for the graft components associated with the main body and branch components can include any material which is suitable for use as a graft in the chosen body lumen. The graft components for the main body and branch(es) can be composed of the same or different materials. The graft components can comprise multiple layers of material that can be the same material or different materials. Although the graft components can have several layers of material, the graft components may have a layer that is formed into a tube (innermost tube) and an outermost layer that is formed into a tube (outermost tube).

Many graft materials are known, particularly known are those that can be used as vascular graft materials. The graft materials can be extruded, coated or formed from wrapped films, or a combination thereof.

Polymers, biodegradable and natural materials can be used for the endoprosthesis for specific applications. Biocompatible materials in particular are contemplated for the various graft components associated with the main body and branch components described herein. In certain instances, the graft components may include an expanded polymer, such as expanded polyethylene (ePE). In some examples, the graft components may include a fluoropolymer, such as a polytetrafluoroethylene (PTFE) polymer or an expanded polytetrafluoroethylene (ePTFE) polymer. In some instances, the graft components may be formed of, such as, but not limited to, a polyethylene, polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Biocompatible materials may be used for the various frame components, or stent components, associated with the main body and branch components described herein. For example, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

### Notice

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An endoprosthesis article of manufacture comprising:
a main body including a crown portion, a hip portion, and an intermediate portion between the crown portion and the hip portion, the crown and hip portions being supported by one or more frame elements and the intermediate portion being unsupported by any frame element;
a first leg extending from the hip portion of the main body; and
a second leg extending from the hip portion of the main body adjacent to the first leg.

2. The endoprosthesis article of claim 1, wherein the intermediate portion is configured for subsequent formation of one or more patient-specific fenestration features.

3. The endoprosthesis article of claim 1, wherein the one or more frame elements have an undulating shape defining a maximum amplitude and the intermediate portion has a height that is at least twice the maximum amplitude, or, optionally, at least five times the maximum amplitude, or optionally at least ten times the maximum amplitude.

4. The endoprosthesis article of claim 3, wherein the one or more patient-specific fenestration features are at pre-selected locations corresponding to one or more abdominal aortic branch vessels of a specific patient.

5. The endoprosthesis article of claim 1, wherein the crown portion is configured to anchor the endoprosthesis in the abdominal aorta at a location superior to the renal arteries.

6. An endoprosthesis comprising:
a main body including a crown portion, a hip portion, and an intermediate portion between the crown portion and the hip portion, the crown and hip portions being supported by one or more frame elements, the intermediate portion having one or more fenestration features associated with the intermediate portion, the one or more fenestration features including an aperture through a graft element of intermediate portion and a port lining extending through the aperture;
a first leg extending from the hip portion of the main body; and
a second leg extending from the hip portion of the main body adjacent to the first leg

7. The endoprosthesis of claim 6, wherein the intermediate portion is unsupported by any frame element.

8. The endoprosthesis of claim 6, wherein the intermediate portion is supported by a frame element formed so as not to obstruct the fenestration feature.

9. The endoprosthesis of claim 6, wherein the intermediate portion is supported by one or more frame elements having an undulating shape that has a varying amplitude and/or frequency to accommodate the fenestration feature, the undulating shape of the one or more frame elements of the intermediate portion being different from the one or more frame elements supporting the crown and the hip portions.

10. The endoprosthesis of claim 6, wherein the port lining is a tubular member formed of a film material and that is free of any frame element.

11. The endoprosthesis of claim 6, wherein the aperture and/or the port lining are reinforced with one or more of the following: polymeric ring elements, fiber elements, wire elements, and combinations thereof.

12. The endoprosthesis of claim 6, wherein the fenestration feature includes a radiopaque marker selected from one or more of the following: a circumferential marker, a dot marker, an impregnated marker, and combinations thereof.

13. A method of making an endoprosthesis comprising:
obtaining patient-specific branch vessel location data; and
forming one or more fenestration features in an unsupported, intermediate portion of an endoprosthesis article of manufacture based upon the patient-specific branch vessel location data.

14. The method of claim 13, further comprising providing the intermediate portion with a frame element supporting the intermediate portion, the frame element being configured so as not to obstruct the one or more fenestration features.

15. The method of claim 13, wherein the endoprosthesis article of manufacture is made prior to the patient-specific branch vessel location data being obtained.

16. The method of claim 13, wherein forming the one or more fenestration features includes forming an aperture through a graft element of intermediate portion and disposing a port lining through the aperture and coupling the port lining to the graft element of the intermediate portion.

17. The method of claim 13, wherein forming the one or more fenestration features includes associating a radiopaque marker selected from one or more of the following with the fenestration feature: a circumferential marker, a dot marker, an impregnated marker, and combinations thereof.

18. The method of claim 13, wherein forming the one or more fenestration features includes forming fenestration features corresponding to at least three aortic branch vessels of a specific patient.

19. The method of claim 18, wherein the aortic branch vessels include a first renal artery, a second renal artery, a mesenteric artery, and/or a celiac trunk artery.

20. The method of claim 13, wherein forming the one or more fenestration features includes reinforcing the aperture with one or more of the following: polymeric ring elements, fiber elements, wire elements, and combinations thereof.
